(19) United States
(12) Patent Application Publication
(10) Pub. No.: US 2024/0398465 A1
(43) Pub. Date: Dec. 5, 2024

(54) MEDICAL SNARE DEVICE

(71) Applicant: Young-Hyun JANG, Gwangju (KR)

(72) Inventor: Young-Hyun JANG, Gwangju (KR)

(21) Appl. No.: 18/694,890

(22) PCT Filed: Nov. 11, 2021
(86) PCT No.: PCT/KR2021/016417

§ 371 Date: Mar. 22, 2024

(30) Foreign Application Priority Data
Sep. 24, 2021 (KR) 10-2021-0126688

(51) Int. Cl. A61B 18/14; A61B 18/00

(52) U.S. Cl. CPC A61B 18/14; A61B 2018/00178; A61B 2018/00196; A61B 2018/00202; A61B 2018/00601; A61B 2018/1475

(57) ABSTRACT

The present invention relates to a medical snare device which can accurately and rapidly cut and remove polyps generated in an organ of the human body, and can minimize bleeding in a part where the polyps are cut and removed, the medical snare device comprising: a main body in which upper/lower guide holes are formed; a rotating body for guiding a polyp removal line and a chemical liquid line; a body entry part for accommodating and protecting the polyp removal line and the chemical liquid line; a polyp removal line operating means for moving the polyp removal line forward/backward; and a chemical liquid line operating means for moving the chemical liquid line forward/backward.

### TECHNICAL FIELD

The present invention relates to a medical snare device capable of accurately and rapidly cutting and removing a polyp developed in an organ of a human body, and minimizing bleeding in a portion in which the polyp is cut and removed.

### BACKGROUND ART

In general, an endoscope is a medical instrument, which is used to directly view an inner surface of a hollow organ having an empty inside, such as a stomach or an esophagus, or an inside of a body, such as a chest cavity or an abdominal cavity, divided into a wide variety of types such as bronchoscope, a laryngoscope, an esophagoscope, a gastroscope/enteroscope, a hysteroscope, a urethroscope, an arthroscope, a cystoscope, and a laparoscope according to their intended use, and very helpful in detection, diagnosis, and treatment of diseases by enabling observation of an internal state of a human body and facilitating tissue resection and secretion collection.

When a polyp is found during a colonoscopy by using the endoscope described above, a procedure for removing the polyp while observing an affected area through the endoscope in a state in which a plurality of surgical instruments are inserted into the human body together with the endoscope may be performed.

In other words, a procedure for first inserting a snare, which is a polyp resection tool, into a human body through an endoscope to allow the snare to make close contact with a found polyp, allowing the snare to protrude from a tube of the endoscope through a manipulation part to resect the polyp by using a wire to which a high-frequency current is supplied, separating the snare from the endoscope, and inserting a separate hemostatic tool into the tube of the endoscope to stop bleeding on one side of the body in which the polyp is removed has been performed as an existing method for removing a polyp.

Regarding the snare described above, there is a need to control a direction of a noose so that a tissue such as a polyp may be inserted into the noose. In other words, proper treatment may be performed only when a tissue of an affected area and a protrusion such as a polyp are inserted into the noose, and the noose is tightened.

Therefore, conventionally, the snare introduced into the tube of the endoscope is configured to transmit a twisting rotational force to the noose introduced into the body through the endoscope by rotating the manipulation part such as a device member and an operating member so as to perform manipulation to twist an insulating tube having flexibility and a wire cord introduced into the insulating tube, so that an operator may adjust the direction of the noose through the manipulation part such as the device member and the operating member while viewing the endoscope.

However, the conventional snare described above had a disadvantage of being inconvenient and difficult to adjust the direction of the noose.

Therefore, the present inventor sought to solve the above problem described above, and Korean Patent Registration No. 10-2262289 (Patent Document which is 1), Korean Patent Registration No. 10-2262289 that has been previously filed and published, discloses a medical snare device shown in FIGS. 1 and 2, in which the medical snare device has a configuration that will be described below.

### BELOW

A medical snare device includes a wire cord 10 installed through a tube 12 and formed at a front end thereof with a noose 11, a rotating body 20 connected to the wire cord to rotate the wire cord, and a handle 30 connected to the rotating body to move the wire cord forward/backward together with the rotating body,
wherein the rotating body 20 includes: a sleeve 21 having an empty inside and an open front side, formed on one side thereof with a long hole 21a, and formed on an opposite side thereof with an electrode hole 21b; a fixing member 22 installed in an open portion of the sleeve, including a high-frequency electrode 22b installed on one side of the fixing member 22 to make contact with the wire cord while the wire cord passes, and formed at a rear center thereof with a guide hole 22a; a lower rotating body 23 connected to a rear side of the fixing member, including a guide 23a fitted to the guide hole to move forward/backward, and including a lower tooth 23b formed on a rear end surface of the guide, formed on one side thereof with a lower vertical part 23b-1, and formed on an opposite side thereof with a lower inclined surface 23b-2 inclined downward from an upper end of the vertical part; an upper rotating body 24 connected to an upper portion of the lower rotating body, including an upper tooth 24b formed on a front end surface of the upper rotating body 24, formed on one side thereof with an upper vertical part 24b-1, and formed on an opposite side thereof with an upper inclined surface 24b-2 inclined downward from an upper end of the vertical part, and including an operating lever 24a installed on one side of the upper rotating body 24; and elastic springs 25 and 26 installed between the fixing member 22 and the lower rotating body 23 and between the upper rotating body and an end of the sleeve, respectively.

Korean Patent Registration No. 10-2262289 (Patent Document 1), which has the above configuration, has a complicated configuration for rotating the noose 11, and has a problem in that the noose may not rotate smoothly.

### DISCLOSURE

### Technical Problem

The present invention has been invented to solve the problems of the relate art as described above, and an object of the present invention is
to provide a medical snare device capable of accurately performing forward/backward movements of a needle, which is configured to inflate a polyp to allow a size of the polyp to be increased by injecting a chemical liquid into the polyp so that a noose may be easily inserted when the polyp developed in an organ is removed, and the noose, which is configured to cut and remove the polyp, capable of precisely rotating the noose, and capable of minimizing bleeding in a portion in which the polyp is cut and removed by the noose, thereby reducing pain of a patient and ensuring rapid recovery.

### Technical Solution

To achieve the object described above, according to the present invention, there is provided a medical snare device comprising:
a main body 100 having a hollow 110 formed inside the main body 100, a fixing member 120 formed at a rear end of the main body 100, an upper guide hole 130 formed in a longitudinal direction on a front side of the fixing member, and a lower guide hole 140 formed in the longitudinal direction on a front side of the upper guide hole;
a rotating body 200 installed at a front end of the main body so as to be able to idle, and configured to rotate while guiding a polyp removal line 320 having a front end in which a noose 310 is installed and a chemical liquid supply line 340 having a front end in which a needle 330 is installed in forward/backward directions;
a body entry part 300 connected to the rotating body to enter a body while the body entry part 300 accommodates the polyp removal line and the chemical liquid supply line so as to allow the noose or the needle to be exposed to an outside of the body entry part 300 or to enter an inside of the body entry part 300 according to operations of a noose operating device 400 and a needle operating device 500;
the noose operating device 400 including a noose moving body 410 in which a rear end of the polyp removal line is installed, and the noose moving body 410 moves forward/backward along the main body, a current supply port 420 formed on one side of the noose moving body, and a noose operating member 430 formed on an opposite side of the noose moving body;
the needle operating device 500 including a needle moving body 510 in which a rear end of the chemical liquid supply line is installed, and the needle moving body 510 moves forward/backward along the main body, a chemical liquid supply port 520 formed on one side of the needle moving body, and a needle operating member 530 formed on an opposite side of the needle moving body; and
a cap 600 installed on one side of the main body in a region in which the needle operating device moves forward/backward so as to be opened and closed so that the needle operating device is able to be separated from and coupled to the main body.

### Advantageous Effects

As described above in the object, the medical snare device according to the present invention, which has the above configuration, is capable of accurately performing forward/backward movements of a needle, which is configured to inflate a polyp to allow a size of the polyp to be increased by injecting a chemical liquid into the polyp so that a noose may be easily inserted when the polyp developed in an organ is removed, and the noose, which is configured to cut and remove the polyp, capable of precisely rotating the noose, and capable of minimizing bleeding in a portion in which the polyp is cut and removed by the noose, thereby reducing pain of a patient and ensuring rapid recovery.

### DESCRIPTION OF DRAWINGS

FIG. 1 is an exploded perspective view for describing a configuration of a conventional medical snare device.

FIG. 2 is an exploded perspective view showing a noose rotation structure in the conventional medical snare device.

FIG. 3 is a perspective view showing a medical snare device according to the present invention.

FIG. 4 is a sectional view showing the medical snare device according to the present invention.

FIG. 5 is a perspective view showing a main body extracted from the medical snare device according to the present invention.

FIGS. 6a and 6b are views showing a configuration of a rotating body extracted from the medical snare device according to the present invention.

FIG. 7 is a perspective view showing a polyp removal line extracted from the medical snare device according to the present invention.

FIG. 8 is a perspective view showing a chemical liquid supply line extracted from the medical snare device according to the present invention.

FIG. 9 is a perspective view showing a noose operating device extracted from the medical snare device according to the present invention.

FIG. 10 is a perspective view showing a needle operating device extracted from the medical snare device according to the present invention.

FIG. 11 is a perspective view showing a bottom surface of a cap extracted from the medical snare device according to the present invention.

FIG. 12 is a sectional view showing a coupling state of a tip of the medical snare device according to the present invention.

FIG. 13 is an enlarged sectional view showing a current supply port extracted from the medical snare device according to the present invention.

FIGS. 14a and 14b are sectional views showing an operational relation between a noose and a needle of the medical snare device according to the present invention.

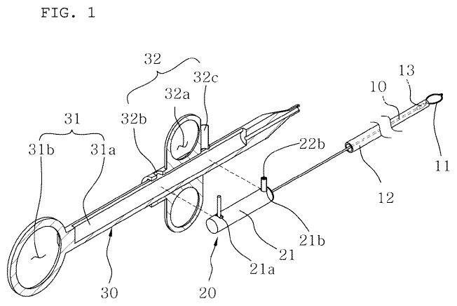

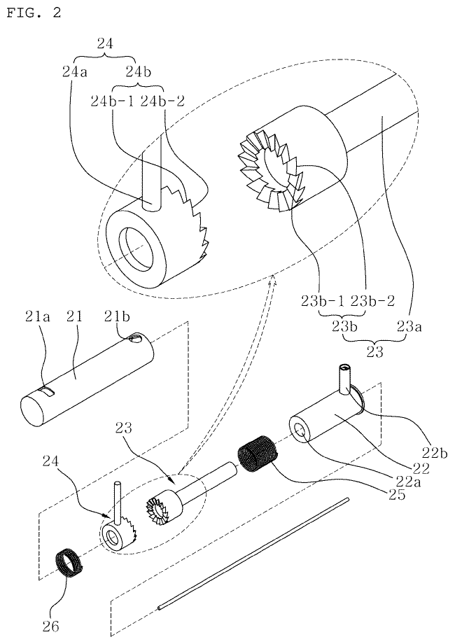

### BEST MODE

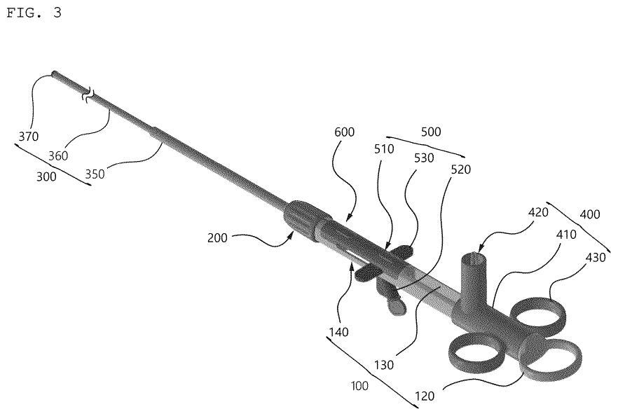

As shown in FIGS. 3 and 4, a medical snare device according to the present invention is a device capable of inflating a polyp developed in an organ inside a body by supplying a chemical liquid to the polyp, inserting and fitting a noose around the inflated polyp, and supplying a high-frequency current to cut the polyp, thereby rapidly and easily removing the polyp while minimizing bleeding.

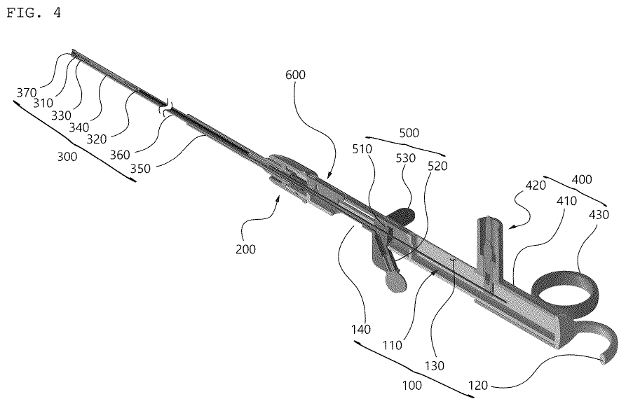

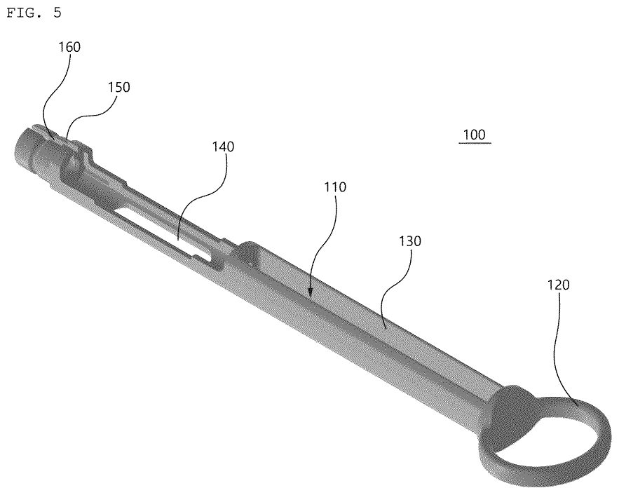

As shown in FIGS. 3 to 5, the medical snare device according to the present invention for rapidly and easily removing the polyp while minimizing the bleeding when the polyp developed in the organ is removed as described above may include a main body 100 having a hollow 110 formed inside the main body 100, a fixing member 120 formed at a rear end of the main body 100, an upper guide hole 130 formed in a longitudinal direction on a front side of the fixing member, and a lower guide hole 140 formed in the longitudinal direction on a front side of the upper guide hole.

The reason why the hollow 110 is formed inside the main body 100 is to mount a noose operating device 400 and a needle operating device 500, which will be described below, and move the mounted noose operating device 400 and the mounted needle operating device 500 forward/backward.

In addition, the reason why the fixing member 120 is to support the main body 100 by inserting a finger of a doctor operating the polyp into the fixing member 120, the reason why the upper guide hole 130 is formed is to restrict a forward/backward movement range of the noose operating device 400, and the reason why the lower guide hole 140 is formed is to restrict a forward/backward movement range of the needle operating device 500.

When the forward/backward movement ranges of the noose operating device 400 and the needle operating device 500 are not restricted, the noose operating device 400 and the needle operating device 500 may move forward more than necessary to allow a noose 310 and a needle 330 to be withdrawn out of a front end of a body entry part 300 more than necessary, so that a polyp removal surgery may become difficult, and a polyp removal portion may be damaged by the noose 310 and the needle 330, and thus it may be preferable to restrict forward movement amounts of the noose operating device 400 and the needle operating device 500.

In addition, although the fixing member 120 is preferably formed in a ring shape to allow the finger of the doctor performing the polyp removal surgery to be fitted and fixed to the fixing member 120, any form that allows the finger of the doctor to fix the main body 100 in addition to the above configuration is possible.

The main body 100 may be formed at a front end thereof with a rotating body coupling part 150 through which a polyp removal line 320 and a chemical liquid supply line 340 pass and on which the rotating body 200 is installed so as to idle.

In addition, the rotating body coupling part 150 may have an incision part 160 for allowing the polyp removal line 320 or the chemical liquid supply line 340 to enter the rotating body coupling part 150.

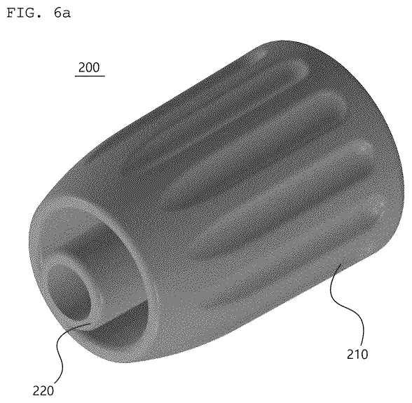

In addition, as shown in FIGS. 6a and 6b, the rotating body 200 according to the present invention may be coupled and installed on the rotating body coupling part 150 formed at the front end of the main body 100 so as to be able to idle to allow the polyp removal line 320 having a front end in which the noose 310 is installed and the chemical liquid supply line 340 having a front end in which the needle 330 is installed to pass through the rotating body 200, and may serve to guide the polyp removal line 320 and the chemical liquid supply line 340 in forward and backward directions.

The reason why the rotating body 200, which serves as described above, is installed on the rotating body coupling part 150 formed at the front end of the main body 100 so as to be able to idle is to easily rotate the noose 310 together with the polyp removal line 320 in an intended direction by gripping the rotating body 200 by a hand and rotating the main body 100 in a desired direction since the noose 310 has be rotated to allow a direction of the noose 310 to match a direction in which the noose 310 may be fitted around the polyp when the noose 310 is fitted around the polyp.

As shown in FIGS. 6a and 6b, the rotating body 200 according to one embodiment, which performs the above operation, may include: a rotating member 210 installed at the front end of the main body 100 so as to idle in place; a sleeve 220 formed integrally with the rotating member inside the rotating member, having an inside through which the polyp removal line 320 and the chemical liquid supply line 340 pass, and having a front end to which an outer tube 350 is coupled; and a guide member 230 formed on a rear side of the sleeve, having an inside through which the polyp removal line 320 and the chemical liquid supply line 340 pass, and having a front end to which an inner tube 360 is connected.

According to the above configuration of the rotating body 200 of one embodiment, it may be preferable that the rear side of the sleeve 220 and a front side of the guide member 230 are closely coupled to each other so as to be formed integrally with each other, thereby interworking with the rotating member 210.

Therefore, when the rotating member 210 is gripped with the hand to rotate the main body 100 in order to rotate the noose 310, the noose 310 may rotate as the polyp removal line 320 rotates together with the main body 100.

However, when the noose 310 is rotated, the noose 310 may be exposed to an outside of the body entry part 300, which will be described below. In addition, when the main body 100 is rotated, the chemical liquid supply line 340 may also rotated.

Regarding the rotation of the main body 100, when the rotating member 200 is gripped by the hand to rotate the fixing member 120, the noose 310 installed at the front end of the polyp removal line 320 may rotate as the main body 100 rotates, and when the noose operating device 400 is rotated, the noose 310 installed at the front end of the polyp removal line 320 may rotate as the main body 100 rotates.

In addition, the sleeve 220 and the guide member 230 are preferably formed in a cylindrical shape having a hollow interior so that the polyp removal line 320 and the chemical liquid supply line 340 may pass smoothly, and an interference-fit scheme in which the sleeve 220 and the guide member 230 are not arbitrarily separated once coupled or a screw-coupling scheme is preferably selected for the coupling of the sleeve 220 and the guide member 230.

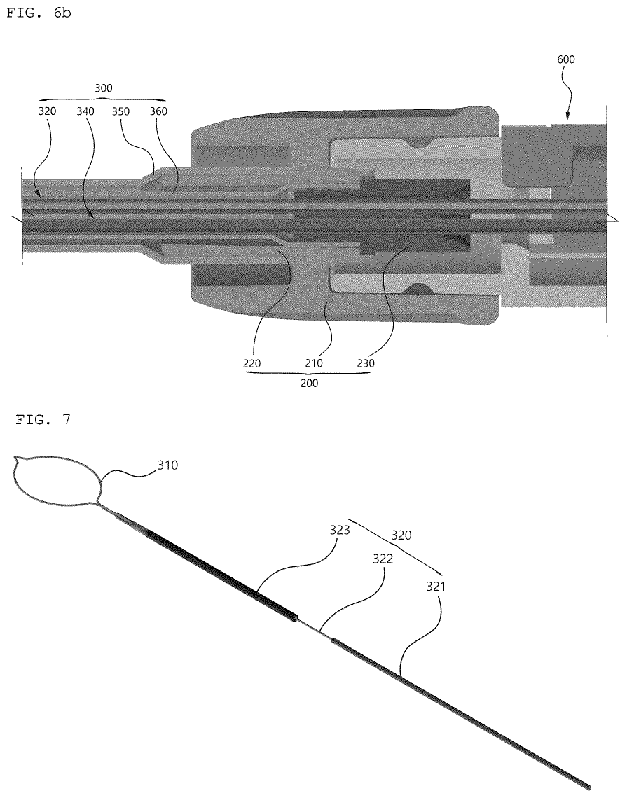

As shown in FIG. 7, the polyp removal line 320 according to one embodiment, which has the front end at which the noose 310 is installed, may include: a steel line 321 having rigidity and a “-” shape, having a hollow formed inside the steel line 321, and installed from the noose operating device 400, which will be described below, to the rotating body 200; a wire 322 through which a current flows, and passing through the hollow formed inside the steel line, in which one end of the wire 322 passing through the steel line 321 is connected to a current supply port 420, and the noose 310 is installed at an opposite end of the wire 322; and a wire protection tube 323 installed on the wire 322 from a portion in which the steel line 321 ends to a portion in which the noose 310 is installed.

According to the above configuration of the polyp removal line 320 of one embodiment, the reason why the steel line 321 has rigidity and a “-” shape and has a hollow formed inside the steel line 321 is to allow the wire 322 to pass through the steel line 321 and to move the steel line 321 forward/backward in a “-” shape without bending or deformation when the steel line 321 moves forward/backward according to an operation of the noose operating device 400.

In addition, the wire 322 is preferably formed of a metal material that may be rotated together with the main body 100 when the main body 100 is rotated, in which the wire 322 formed of the metal material has to be formed of a metal material that is bendable and has elasticity to recover after bending.

The reason why the above configuration is provided is to allow the body entry part 300 to be easily bent to enter along a curve of an organ when the body entry part 300 enters the organ of a human body.

Meanwhile, the wire 322 formed of the metal material having the elasticity may be closely fitted and coupled to the wire protection tube 323 formed of a resin material. The reason why the above configuration is provided is to prevent deformation of the wire 322 by allowing the wire 322 to be rotated together with the main body 100 when the main body 100 is rotated, so that a rotational force of the main body 100 may be completely transmitted to the wire 322, and thus the noose 310 may rotate precisely according to the accurate rotation of the wire 322, and to prevent the current flowing through the wire 322 from being transmitted to the chemical liquid supply line 340.

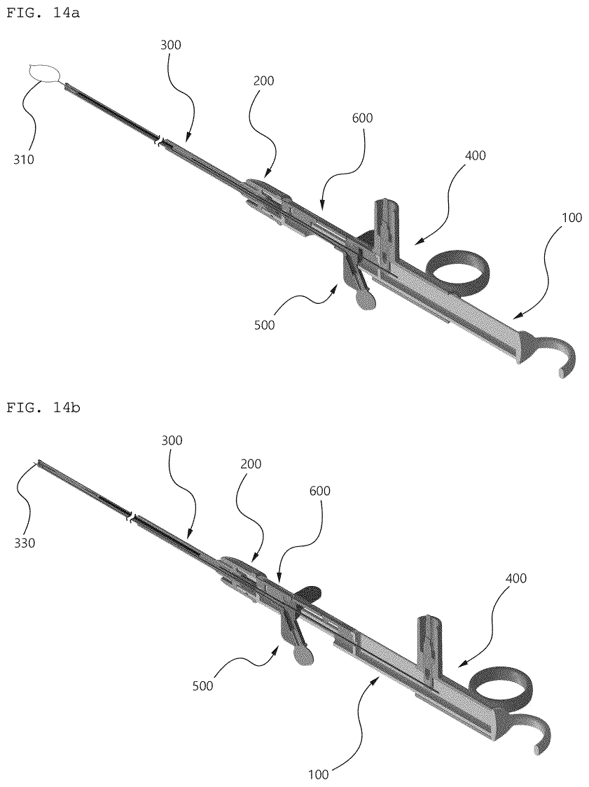

The noose 310 according to one embodiment is preferably formed as a core line connected to a portion in which the wire 322 ends so as to form an arc shape when the noose 310 is exposed to the outside of the body entry part 300 as shown in FIG. 14a, and to allow the arc shape to overlap in a straight line shape when the noose 310 enters the inside of the body entry part 300 as shown in FIG. 14b, in which the core line may be formed of a metal material having elasticity and through which a current flows.

The reason why the noose 310 having the configuration described above is formed as the core line (a steel line that is thinner than the wire 322) is to easily cut the polyp when the polyp is tied up with the noose 310 and pulled.

In addition, the reason why the noose 310 forms an arc shape when the noose 310 is exposed to the outside of the body entry part 300 and the arc shape overlaps in a straight line shape when the noose 310 enters the inside of the body entrance part 300 is to form an arc shape, which is an original shape, capable of fitting and coupling the polyp when the noose 310 is exposed to the outside of the body entry part 300, and to allow the arc shape to overlap in a straight line shape by elasticity when the noose 310 enters the inside of the body entry part 300 since the noose 310 has elasticity.

In addition, as shown in FIG. 8, the chemical liquid supply line 340 according to the present invention may include: a steel line 341 having rigidity and a “-” shape, having a hollow formed inside the steel line 341, and installed from the needle operating device 500 to the rotating body 200; a chemical liquid tube 342 passing through the inside of the steel line, and connected to the chemical liquid supply port 520, in which the chemical liquid tube 342 extends from a portion in which the steel line 341 ends to a portion in which the needle 330 is installed; and a chemical liquid protection tube 343 installed on the extending chemical liquid tube.

According to the above configuration of the chemical liquid supply line 340, the reason why the steel line 341 has rigidity and a “-” shape and has a hollow formed inside the steel line 341 is to allow the chemical liquid tube 342 to pass through the steel line 341 and to move the steel line 341 forward/backward in a “-” shape, that is, in a horizontal direction without bending or deformation when the steel line 341 moves forward/backward according to an operation of the needle operating device 500, the chemical liquid tube 342 may supply a chemical liquid injected from the chemical liquid supply port 520 to the needle 330, and the chemical liquid protection tube 343 may serve to accommodate and protect the chemical liquid tube 342.

Meanwhile, the body entry part 300 according to the present invention may be connected to the rotating body 200 to enter a body while the body entry part 300 accommodates the polyp removal line 320 and the chemical liquid supply line 340 so as to serve to allow the noose 310 or the needle 330 to be exposed to the outside of the body entry part 300 or to enter the inside of the body entry part 300 according to operation directions of the noose operating device 400 and the needle operating device 500.

An operation of allowing the noose 310 or the needle 330 to be exposed to the outside of the body entry part 300 or to enter the inside of the body entry part 300 as described above may be performed such that the needle 330 may enter the inside of the body entry part 300 when the noose 310 is exposed to the outside of the body entry part 300 as shown in FIG. 14a, and the noose 310 may enter the inside of the body entry part 300 when the needle 330 is exposed to the outside of the body entry part 300 as shown in FIG. 14b.

The body entry part 300 according to one embodiment, which performs the above operation, may include: an inner tube 360 through which the noose 310 and the needle 330 enter and exit while the inner tube 360 accommodates the polyp removal line 320 and the chemical liquid supply line 340; and an outer tube 350 for accommodating a portion of the inner tube 360 at a front end of the rotating body 210.

According to the above configuration of the body entry part 300 of one embodiment, the inner tube 360 may serve to accommodate and protect the polyp removal line 320 and the chemical liquid supply line 340, to allow the polyp removal line 320 and the chemical liquid supply line 340 to easily enter the body, and not to cause harm to the body after the entry, and the outer tube 350 may serve to protect a connection portion between the rotating body 200 and the inner tube 360 from being bent.

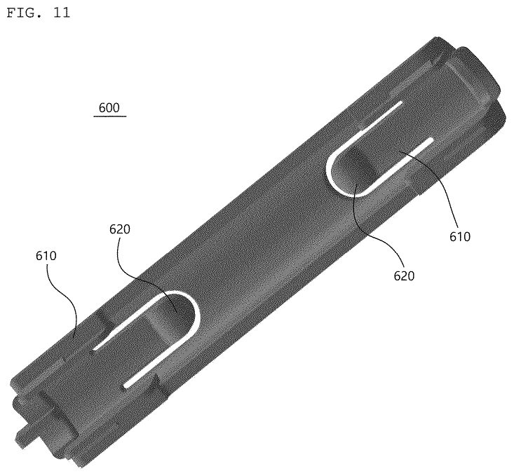

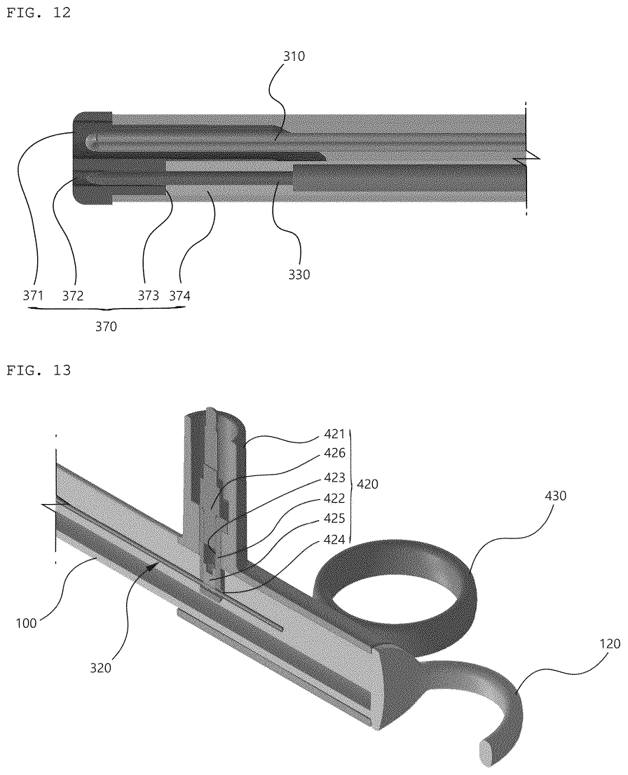

In addition, as shown in FIG. 12, the body entry part 300 according to the present invention may be provided at a front end thereof with a tip 370 including: a noose entry/exit hole 371 and a needle entry/exit hole 372 through which the noose 310 and the needle 330 enter and exit, respectively; a latching sill 373 formed on a rear side of the needle entry/exit hole; and a needle guide 374 installed in the needle entry/exit hole 372 formed on the rear side thereof with the latching sill and configured to guide the needle 300.

According to the above configuration of the tip 370, the reason why the needle guide 374 is installed is to allow the needle 330 to be straightly withdrawn out in a “-” shape when the needle 330 enters and exits the front end of the body entry part 300, and the reason why the latching sill 373 is formed is to allow a front end of the chemical liquid tube 342 to be latched to the latching sill 373 so that only a predetermined amount of the needle 330 is withdrawn out of the body entry part 300, and thus the chemical liquid may be stably injected when the chemical liquid is injected into a the polyp.

In addition, the noose entry/exit hole 371 and the needle entry/exit hole 372 may have diameters that allow the noose 310 and the needle 330 to enter and exit smoothly, and the noose entry/exit hole 371 may be larger than the needle entry/exit hole 372 to facilitate the entry and exit of the noose 310.

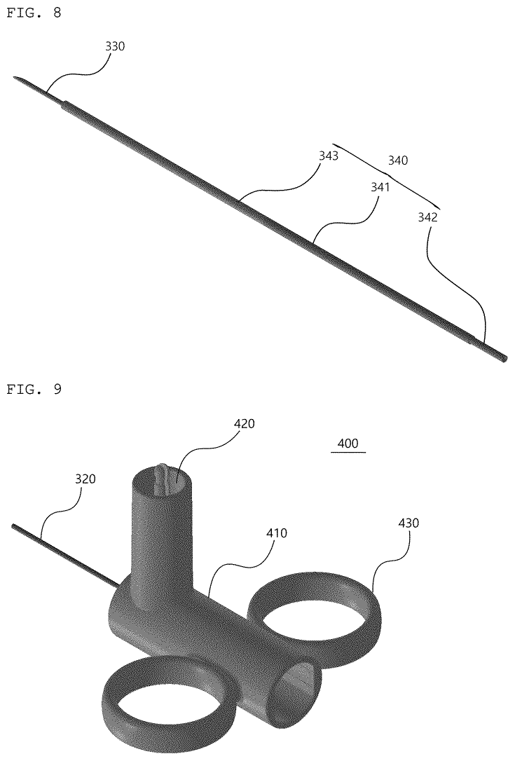

As shown in FIG. 9, the noose operating device 400 according to the present invention may include: a noose moving body 410 in which a rear end of the polyp removal line 320 is installed, and the noose moving body 410 moves forward/backward along the main body 100; a current supply port 420 installed on one side of the noose moving body; and a noose operating member 430 formed on an opposite side of the noose moving body.

The noose operating device 400 having the above configuration may perform an operation of moving the polyp removal line 320 while moving in forward/backward directions when the noose operating device 400 moves forward/backward in the longitudinal direction of the main body 100, so that the noose 310 may be exposed to the outside of the body entry part 300 or may enter the inside of the body entry part 300.

According to the above configuration of the noose operating device 400, the noose moving body 410 may be provided in the form of a pipe having a hollow formed inside the pipe, fitted and coupled to the main body 100 to accommodate the main body 100, and configured to move in the longitudinal direction of the main body 100, and the noose moving body 410 may be formed in a shape matching an external shape of the main body 100 so that the noose moving body 410 may move stably along the main body 100.

A hollow portion of the noose moving body 410 described above may have a circular shape, an oval shape, or a triangular or more shape, and the external shape of the main body 100 in a region in which the noose moving body 410 is coupled and moved may also be a circular shape, an oval shape, or a triangular or more shape that matches the shape of the hollow portion of the noose moving body 410.

However, each of the shape of the hollow portion of the noose moving body 410 and the external shape of the main body 100 in the region in which the noose moving body 410 is coupled and moved is preferably an oval shape or a triangular or more shape, and the reason why the above configuration is provided is to allow the noose moving body 410 to move forward/backward in a straight direction without being rotated while the noose moving body 410 moves along the main body 100.

In addition, noose operating members 430 are preferably formed in a ring shape on both sides of the noose moving body to allow two fingers to be fitted, respectively, and the reason why the above configuration is provided is to stably move the noose moving body 410 forward/backward.

In addition, as shown in FIG. 13, the current supply port 420 may include: a current supply body 421 having a hollow formed inside the current supply body 421; a fixing guide member 422 inserted into an inner lower portion of the current supply body 421, and having a screw hole 423 for guiding fixing of the rear end of the polyp removal line 320; a fixing screw 425 formed at a lower end of the fixing guide member 422, having a fixing hole 424 to which a steel line 321 having a “-” shape is fitted and fixed, and coupled to the screw hole 423; and a current supply piece 426 installed in the screw hole 423 on a rear side of the fixing screw 425, and configured to supply a current.

According to the above configuration of the current supply port 420, the fixing guide member 422 and the current supply piece 426 may be installed in the current supply body 421, the fixing screw 425 may be installed in the screw hole 423 of the fixing guide member 422, a portion of a rear side of the polyp removal line 320 may be fitted and fixed to the fixing hole 424, and the fixing screw 425 may fixe the rear side of the polyp removal line 320 fitted to the fixing hole 424. In addition, the current supply piece 426 may supply an external current so as to supply the current to the noose 310 through the polyp removal line 320.

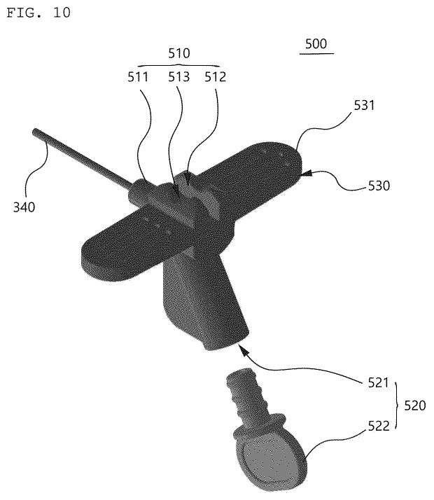

As shown in FIG. 10, the needle operating device 500 according to the present invention may include: a needle moving body 510 in which a rear end of the chemical liquid supply line 340 is installed, and the needle moving body 510 moves forward/backward along the main body 100; a chemical liquid supply port 520 formed on one side of the needle moving body; and a needle operating member 530 formed on an opposite side of the needle moving body.

The needle operating device 500 having the above configuration may perform an operation of moving the chemical liquid supply line 340 while moving in forward/backward directions when the needle operating device 500 moves forward/backward in the longitudinal direction of the main body 100, so that the needle 330 may be exposed to the outside of the body entry part 300 or may enter the inside of the body entry part 300.

According to the above configuration of the needle operating device 500, the needle moving body 510 may include: a fixing member 511 to which a steel line 341 having a “-” shape, which is a component of the chemical liquid supply line 340, is coupled and fixed; and a guide part 512 formed on an upper portion of the fixing member, and configured to guide the polyp removal line 320, thereby allowing the fixation of the chemical liquid supply line 340 and the forward/backward movements of the polyp removal line 320 to be smoothly performed.

In addition, a seating groove 513 in which a protrusion part 620 of a control piece 610 formed on a cap 600, which will be described below, is seated may be formed on the guide part 512.

According to the above configuration of the needle moving body 510, the fixing member 511 may fix a rear end of the steel line 341 having a “-” shape, the guide part 512 may serve to guide the polyp removal line 320 so that the polyp removal line 320 may smoothly move forward/backward without being caught in the needle moving body 510 when the polyp removal line 320 moves forward/backward, and the seating groove 513 is a portion in which the protrusion part 620 of the control piece 610 formed on the cap 600, which will be described below, is seated to control the needle operating device 500 not to operate arbitrarily.

In addition, the chemical liquid supply port 520 may include: an insertion hole 521 protruding outward from one side of the needle moving body 510, and into which an inlet of a syringe may be inserted to inject a chemical liquid; and a stopper 522 provided in the insertion hole 521 so as to be opened to supply the chemical liquid when the chemical liquid is supplied, and closed to have airtightness after the chemical liquid is supplied.

According to the above configuration of the chemical liquid supply port 520, the insertion hole 521 may be a portion into which the inlet of the syringe is inserted to supply the chemical liquid to the needle 330 through the chemical liquid supply line 340, and the stopper 522 may be a component configured to prevent a foreign substance from being introduced into the insertion hole 521 when the chemical liquid is not supplied.

In addition, the needle operating member 530 may include a handle 531, which is formed on both sides of the needle moving body 510 and may be gripped by a hand, and the reason why the needle operating member 530 is formed on both sides of the needle moving body 510 is to move the needle moving body 510 by gripping the needle moving body 510 with a hand selected from a right hand or a left hand.

As shown in FIGS. 3 and 11, the cap 600 according to the present invention may be installed on one side of the main body 100 in a region in which the needle operating device 500 moves forward/backward so as to be opened and closed so that the needle operating device 500 may be separated from and coupled to the main body 100.

The reason why the cap 600 is separated from and coupled to the main body 100 is that one side of the main body 100 has to be opened since the needle operating device 500 has to be fitted and coupled to a hollow portion 110 of the main body 100.

Therefore, according to the present invention, the cap 600 may be formed on one side of the main body 100 in the region in which the needle operating device 500 moves forward/backward so as to be opened and closed.

The cap 600 may be formed on front/rear sides thereof with a control piece 610 for controlling the needle operating device 500 to not operate arbitrarily when the needle 330 is completely exposed to the outside of the body entry part 300 and when the needle 330 completely enters the inside of the body entry part 300 according to the operation of the needle operating device 500, so that an arbitrary operation of the needle operating device 500 may be controlled by the control piece 610.

The control piece 610 may be formed on an inner surface thereof with a protrusion part 620 for applying a pressure to the needle operating device 500, so that the protrusion part 620 formed on one side may be seated in the seating groove 513 formed on one side to prevent the needle operating device 500 from moving arbitrarily when the needle 330 is completely exposed to the outside of the body entry part 300, and the protrusion part 620 formed on an opposite side may be seated in the seating groove 513 formed on an opposite side to prevent the needle operating device 500 from moving arbitrarily when the needle 330 completely enters the inside of the body entry part 300, and thus stability may be ensured during the polyp removal surgery.

As shown in FIG. 14a, when the noose operating device 400 moves forward from the main body 100, the polyp removal line 320 may move forward so that the noose 310 may be exposed to the outside of the body entry part 300, when the needle operating device 500 moves forward from the main body 100, the chemical liquid supply line 340 may move forward so that the needle 330 may be exposed to the outside of the body entry part 300, when the needle 330 is exposed, the noose operating device 400 may move backward from the main body 100 so that the noose 310 may enter the inside of the body entry part 300, and when the noose 310 is exposed, the needle operating device 500 may move backward from the main body 100 so that the needle 330 may enter the inside of the body entry part 300.

As described above in the effect, the medical snare device according to the present invention described above is capable of accurately performing forward/backward movements of a needle, which is configured to inflate a polyp to allow a size of the polyp to be increased by injecting a chemical liquid into the polyp so that a noose may be easily inserted when the polyp developed in an organ is removed, and the noose, which is configured to cut and remove the polyp, capable of precisely rotating the noose, and capable of minimizing bleeding in a portion in which the polyp is cut and removed by the noose, thereby reducing pain of a patient and ensuring rapid recovery.

1. A medical snare device comprising:
a main body having a hollow formed inside the main body, a fixing member formed at a rear end of the main body, an upper guide hole formed in a longitudinal direction on a front side of the fixing member, and a lower guide hole formed in the longitudinal direction on a front side of the upper guide hole;
a rotating body installed at a front end of the main body so as to be able to idle, and configured to rotate while guiding a polyp removal line having a front end in which a noose is installed and a chemical liquid supply line having a front end in which a needle is installed in forward/backward directions;
a body entry part connected to the rotating body to enter a body while the body entry part accommodates the polyp removal line and the chemical liquid supply line so as to allow the noose or the needle to be exposed to an outside of the body entry part or to enter an inside of the body entry part depending on operations of a noose operating device and a needle operating device;
the noose operating device including a noose moving body in which a rear end of the polyp removal line is installed, and the noose moving body moves forward/backward along the main body, a current supply port formed on one side of the noose moving body, and a noose operating member formed on an opposite side of the noose moving body;
the needle operating device including a needle moving body in which a rear end of the chemical liquid supply line is installed, and the needle moving body moves forward/backward along the main body, a chemical liquid supply port formed on one side of the needle moving body, and a needle operating member formed on an opposite side of the needle moving body; and
a cap installed on one side of the main body in a region in which the needle operating device moves forward/backward so as to be opened and closed so that the needle operating device is able to be separated from and coupled to the main body.

2. The medical snare device of claim 1, wherein the cap is formed on front/rear sides thereof with a control piece having elasticity for controlling the needle operating device to not operate arbitrarily when the needle is completely exposed to the outside of the body entry part and when the needle completely enters the inside of the body entry part according to the operation of the needle operating device.

3. The medical snare device of claim 2, wherein the control piece is formed on an inner surface thereof with a protrusion part for applying a pressure to the needle operating device.

4. The medical snare device of claim 1, wherein the fixing member is formed at the rear end of the main body in a ring shape to allow a finger to be fitted and fixed to the fixing member.

5. The medical snare device of claim 1, wherein the rotating body includes: a rotating member installed at the front end of the main body so as to idle in place; a sleeve formed integrally with the rotating member inside the rotating member, having an inside through which the polyp removal line and the chemical liquid supply line pass, and having a front end to which an outer tube is coupled; and a guide member formed on a rear side of the sleeve, having an inside through which the polyp removal line and the chemical liquid supply line pass, and having a front end to which an inner tube is connected.

6. The medical snare device of claim 1, wherein the polyp removal line includes: a steel line having rigidity and a “-” shape, having a hollow formed inside the steel line, and installed from the noose operating device to the rotating body; a wire through which a current flows, and passing through the hollow formed inside the steel line, in which one end of the wire passing through the steel line is connected to the current supply port, and the noose is installed at an opposite end of the wire; and a wire protection tube installed on the wire from a portion in which the steel line ends to a portion in which the noose is installed.

7. The medical snare device of claim 1, wherein the noose is formed as a core line having elasticity, through which a current flows, and connected to a portion in which a wire ends so as to form an arc shape when the noose is exposed to the outside of the body entry part, and to allow the arc shape to overlap in a straight line shape when the noose enters the inside of the body entry part.

8. The medical snare device of claim 1, wherein the chemical liquid supply line includes: a steel line having rigidity and a “-” shape, having a hollow formed inside the steel line, and installed from the needle operating device to the rotating body; a chemical liquid tube passing through the inside of the steel line, and connected to the chemical liquid supply port, in which the chemical liquid tube extends from a portion in which the steel line ends to a portion in which the needle is installed; and a chemical liquid protection tube installed on the extending chemical liquid tube.

9. The medical snare device of claim 1, wherein the body entry part includes: an inner tube through which the noose and the needle enter and exit while the inner tube accommodates the polyp removal line and the chemical liquid supply line; and an outer tube for accommodating a portion of the inner tube at a front end of the rotating body.

10. The medical snare device of claim 1, wherein the body entry part is provided at a front end thereof with a tip through which the noose and the needle enter and exit, the tip has a noose entry/exit hole and a needle entry/exit hole, the needle entry/exit hole is formed on a rear side thereof with a latching sill, and a needle guide for guiding the needle is installed in the needle entry/exit hole formed on the rear side thereof with the latching sill.

11. The medical snare device of claim 1, wherein the noose moving body is provided in a form of a pipe having a hollow formed inside the pipe, fitted and coupled to the main body to accommodate the main body, and configured to move in the longitudinal direction of the main body.

12. The medical snare device of claim 1, wherein noose operating members are formed in a ring shape on both sides of the noose moving body to allow two fingers to be fitted, respectively.

13. The medical snare device of claim 1, wherein the current supply port includes: a current supply body having a hollow formed inside the current supply body; a fixing guide member inserted into an inner lower portion of the current supply body, and having a screw hole for guiding fixing of the rear end of the polyp removal line; a fixing hole formed at a lower end of the fixing guide member, and to which a steel line having a “-” shape is fitted and fixed; a fixing screw screw-coupled to the screw hole, and configured to fix the rear end of the polyp removal line; and a current supply piece installed in the screw hole on a rear side of the fixing screw, and configured to supply a current.

14. The medical snare device of claim 1, wherein the needle moving body includes: a fixing member to which a steel line having a “-” shape, which is a component of the chemical liquid supply line, is coupled and fixed; a guide part formed on an upper portion of the fixing member, and configured to guide the polyp removal line; and a seating groove formed on an upper portion of the guide part, and in which a protrusion part of a control piece which is a component of the cap, is seated.

15. The medical snare device of claim 1, wherein the chemical liquid supply port includes: an insertion hole protruding outward from one side of the needle moving body, and into which an inlet of a syringe is able to be inserted to inject a chemical liquid; and a stopper provided in the insertion hole so as to be opened to supply the chemical liquid when the chemical liquid is supplied, and closed to have airtightness after the chemical liquid is supplied.

16. The medical snare device of claim 1, wherein the needle operating member includes a handle, which is formed on both sides of the needle moving body and able to be gripped by a hand.

17. The medical snare device of claim 6, wherein the noose is formed as a core line having elasticity, through which a current flows, and connected to a portion in which the wire ends so as to form an arc shape when the noose is exposed to the outside of the body entry part, and to allow the arc shape to overlap in a straight line shape when the noose enters the inside of the body entry part.
